(19) United States
(12) Patent Application Publication
(10) Pub. No.: US 2005/0191337 A1
(43) Pub. Date: Sep. 1, 2005

(54) Patch with a magnetic field effect

(75) Inventor: Jean-Louis H. Gueret, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL S.A.

(21) Appl. No.: 11/060,811

(22) Filed: Feb. 18, 2005

Related U.S. Application Data
(63) Continuation-in-part of application No. 09/541,464, filed on Mar. 31, 2000.

(30) Foreign Application Priority Data
Mar. 31, 1999 (FR) 99 04043

(51) Int. Cl. A61L 15/16

(52) U.S. Cl. 424/448

(57) ABSTRACT

A patch may include at least one polymer matrix having a surface intended to be placed in contact with the skin. The surface may be one of adhesive and configured to become adhesive. The polymer matrix may include at least one active substance effective for acting on the skin. The at least one active substance may include a solid active substance. The polymer matrix may be anhydrous. The patch may include a plurality of magnetic particles in the polymer matrix. The plurality of magnetic particles may be permanently oriented over at least a portion of the patch, and the patch may be in dry form prior to use.

This application is a continuation-in-part of U.S. patent application Ser. No. 09/541,464, filed Mar. 31, 2000, the subject matter of which is incorporated herein by reference.

The present invention relates to a patch for temporary application to the skin to provide a cosmetic and/or a pharmaceutical treatment.

Conventional patches that are applied to the skin comprise a support sheet coated on one face with a layer forming a reservoir, referred to as the matrix. This matrix generally contains one or more active substances capable of diffusing into the skin and/or acting on the skin.

In the case of a skin treatment using active agents to achieve deep-down treatments or reactions, the efficacy of the treatment may, aside from the choice of a suitable active agent or agents, depend on the ability of the active agents to penetrate into the skin. Such an ability depends not only on the intrinsic properties of the active agent but also on the skin itself. To this end, it is possible to treat the skin in order to promote its receptivity to active agents.

It has previously been shown that a magnetic field applied to the skin can have a beneficial effect. For example, the circulation in the capillaries, as well as on various aspects of metabolism are improved. It is also known that improving the circulation in the capillaries can, among other things, play a role in weight reduction, or alternatively can promote the penetration of active agents, in particular cosmetic agents. One of the problems that to date has not been solved relates to the practical implementation of such a magnetic field in combination with the application of the active agent or agents, without creating any time-intensive and laborious manipulations for the user. Attempts have thus been made, without much success, to incorporate magnetic particles into creams, pastes, oils, lotions, and the like.

German utility model DE 86 10 769.0 suggests incorporating, into a cream intended to form a beauty or care mask, small magnets having a magnetic moment with at least one north pole and one south pole. The cream is inorganic, for example, based on clay, petroleum jelly or plaster. The size of the magnets preferably ranges from 0.5 mm to 30 mm, and more preferably from 2 mm to 10 mm.

DE-A-36 19 987 discusses a major problem suffered by the mask described in DE 86 10 769.0, mentioned above. This problem relates to the fact that, on account of the permanent magnetization of the magnets, they have a tendency to attract or repel each other, and thus form packets. In the resulting preparation, therefore, the magnets are not dispersed homogeneously. The results of the application are unsatisfactory. To solve this problem, DE-A-36 19 987 proposes bonding the magnets to a support and then coating the support with the cosmetic preparation. The user thus applies the preparation as deposited on a support. Such a solution suffers mainly due to the fact that when the inorganic matrix is based on plaster or clay, for example, it dries out and hardens a certain amount of time after it has been applied, creating a relatively pronounced sensation of discomfort. The rigidity of the assembly is enhanced by the presence of the relatively large magnets bonded to the support. In addition, the magnets are not distributed uniformly in the preparation, and this may be detrimental in terms of efficacy.

It also is known, in particular from DE-A-197 15 478, to introduce magnetic particles, particularly ferrites, into a cosmetic or dermatological composition. The particles that are introduced, however, are not permanently magnetized.

Thus, one of the objects of the invention is to be able to prepare a structure, for example in the form of a cosmetic or dermatological treatment patch, formed from a matrix in which permanently oriented magnetic particles are dispersed homogeneously.

It should be understood that the invention could be practiced without performing one or more of the preferred objects and/or advantages described above. Other objects will become apparent from the detailed description which follows.

The present invention includes the discovery that such a magnetic field effect can be obtained simply and effectively by incorporating the active agent(s) to be used into the polymer matrix of a patch, which is preferably adhesive, and by dispersing in the matrix an amount of magnetic particles, which are permanently oriented by placing the patch in a magnetic field after setting the matrix. For example, the magnetic particles may be permanently immobilized in the polymer matrix, and/or the magnetic particles may generate a magnetic field that is permanently oriented. The skin thus simultaneously comes into contact with one or more active agents and with the permanently oriented magnetic particles, so as to promote the penetration of the active agents into the skin.

Thus, one aspect of the present invention includes a patch comprising at least one polymer matrix. One surface of the matrix is intended to be placed in contact with the skin, and this surface is or configured to become adhesive, after moistening for example. The polymer matrix comprises at least one active substance effective for treating or reacting with the skin, for example cosmetically treating or reacting with the skin. At least one magnetic particle is dispersed in the matrix and permanently oriented over at least a portion of the area of the patch. The polymer matrix is preferably anhydrous or hydrophobic. Preferably, a plurality of magnetic particles are homogeneously dispersed in the matrix and these particles are permanently oriented over at least a portion of the area of the patch. For example, the polymer matrix may include a gel, and the magnetic particles may be mixed into the gel such that they are distributed in the gel and immobilized in the gel.

Once the matrix has been at least partially polymerized, preferably by techniques such as crosslinking, polyaddition, or polycondensation, the matrix forms a set structure in which the magnetic particles are immobilized. In this configuration, the particles of all or a portion of the patch can be subjected to a magnetic field, so as to be permanently magnetized. The particles thus set in the polymer matrix remain uniformly dispersed in the matrix and retain their magnetic orientation. When applied to the skin, the effects are thus uniform over the entire magnetized area of the patch.

Thus, during application, the magnetic field lines generated by the permanently oriented magnetic particles have a beneficial action, in particular on the oxygenation of the skin and on the circulation in the capillaries, thus promoting the penetration of the active agents into the skin and reinforcing their efficacy. For example, the strength of the magnetic field may be relatively low (e.g., ranging from about 0.3 gauss to about 10 gauss), for example, in order to be compatible with direct application to the skin. As a result, the time during which the patches according to the invention are applied can be substantially reduced. The concentrations of certain active agents considered to be aggressive to the skin can be appreciably reduced. In addition, the magnetic particles can themselves act as an active substance, since the magnetic field they generate can, by acting on the circulation in the capillaries, have other effects such as weight reduction, for example.

The magnetic particles preferably are particles of ferrite, for example based on zinc and manganese. Alternatively, particles sold under the trade name MagaBeads® by the company Cortex Biochem are used.

The percentage by volume of magnetic particles in the polymer matrix can range from about 0.2% to about 80%, and preferably from about 1% to about 30%, and even more preferably from about 2% to about 15%.

The particles can be coated with coatings such as, for example, polyurethane, epoxy resin, polyester, polyamide or cyanoacrylate, or other similar like substances, which may serve to protect the particles from humidity. Such a coating may assist in protecting the magnetic particles when they are placed in the presence of water, in particular under the effect of perspiration of the skin.

The magnetic particles can be oriented such that the magnetic field lines generated extend from a first face of the patch to a second face opposite the first face over at least a portion of the area of the patch. Alternatively, the magnetic particles are oriented such that the generated magnetic field lines extend from a first edge of the patch to a second edge opposite the first edge over at least a portion of the area of the patch. Other arrangements or combinations also can be obtained, for example by multiplying the passages in identical or different magnetic fields, or by varying the characteristics of the magnetization bed used.

The active substance preferably is chosen from weight-reducing agents, cleansing agents, antioxidants, free-radical scavengers, moisturizers, depigmenting agents, liporegulators, anti-acne agents, anti-seborrhoeic agents, anti-ageing agents, softeners, anti-wrinkle agents, keratolytic agents, anti-inflammatory agents, refreshing agents, cicatrizing agents, vascular protectors, antibacterial agents, antifungal agents, antiperspirants, deodorants, skin conditioners, desensitizers, immunomodulators and nourishing agents, moisture absorbers (cotton, polyacrylate) or sebum absorbers (Orgasol), or a combination of these substances. However, almost any active substance can be selected depending on the desired reaction.

The at least one active substance preferably comprises a water soluble agent, for example, a water-soluble active substance chosen from enzymes; antibiotics; components with a tensioning effect; sugars and sugar derivatives; urea; oligopeptides; water-soluble plant extracts; yeasts; protein hydrolysates; mucopolysaccharides; vitamins B₂, B₆, H, and PP; panthenol; allantoin; hydroquinone; _-hydroxy acids, polyhydroxy acids, amino acids, hyaluronic acid, folic acid, acetylsalicylic acid, glycyrrhetic acid, kojic acid, ascorbic acid and biologically compatible salts thereof.

In addition, the at least one active substance can be a liposoluble active substance chosen from D-_-tocopherol, DL-_-tocopherol, D-_-tocopheryl acetate, DL-_-tocopheryl acetate, ascorbyl palmitate, vitamin F and vitamin F glycerides, D vitamins, vitamin D2, vitamin D3, retinol, retinol esters, retinyl palmitate, retinyl propionate, _-carotene, D panthenol, farnesol, farnesyl acetate; jojoba oil and blackcurrant oil rich in essential fatty acids; keratolytic agents such as salicylic acid, its salts and its esters, 5-n-octanoylsalicylic acid and its esters, alkyl esters of _-hydroxy acids such as citric acid, lactic acid or glycolic acid; asiatic acid, madecassic acid, asiaticoside, total extract of Centella asiatica, _-glycyrrhetinic acid, _-bisabolol, ceramides such as 2-oleoylamino-1,3 octadecane; phytanetriol, milk sphingomyelin, phospholipids of marine origin which are rich in polyunsaturated essential fatty acids, ethoxyquine; extract of rosemary, extract of balm, quercetin, extract of dried microalgae, steroidal anti-inflammatory agents.

Such active agents can be incorporated in dissolved form in oils, used alone or in combination, among which mention may be made of: oils of animal, plant or mineral origin, and in particular animal or plant oils formed by fatty acid esters of polyols, in particular liquid triglycerides, for example sunflower oil, corn oil, soybean oil, marrow oil, grape pip oil, sesame oil, hazelnut oil, pistachio oil, apricot oil, almond oil or avocado oil; fish oils, glyceryl tricaprocaprylate, or plant or animal oils of formula R₁COOR₂ in which R₁ represents a higher fatty acid residue, preferably comprising from 7 to 19 carbon atoms and R₂ represents a branched hydrocarbon-based chain preferably comprising from 3 to 20 carbon atoms, for example purcellin oil; wheatgerm oil, beauty-leaf oil, sesame oil, coriander oil, safflower oil, passion flower oil, musk rose oil, macadamia oil, fruit (grape, blackcurrant, orange, kiwi) pip oil, rape seed oil, coconut oil, groundnut oil, evening primrose oil, palm oil, castor oil, flax oil, jojoba oil, chia oil, olive oil or cereal germ oil such as wheatgerm oil, rice bran oil or karite butter; acetylglycerides; alkyl or polyalkyl octanoates, decanoates or ricinoleates; fatty acid triglycerides; glycerides; liquid paraffin, liquid petroleum jelly, perhydrosqualene; fatty alcohols (stearyl alcohol, cetyl alcohol) and fatty acids (stearic acid) and esters thereof; poly(C₁-C₂₀)alkylsiloxanes and in particular those containing trimethylsilyl end groups, preferably those with a viscosity of less than about 0.06 m²/s, among which mention may be made of linear polydimethylsiloxanes and alkylmethylpolysiloxanes such as cetyldimethicone (CTFA name). Moreover, partially fluorinated hydrocarbon-based oils or perfluoro oils, and in particular perfluoropolyethers and perfluoroalkanes, also may be used.

The oily phase, i.e. the droplets of oil dispersed in the polymer matrix, can be present in a proportion which can range from about 0.1% to about 30% by weight relative to the total weight of the composition. Preferably, this percentage is from about 5% to about 25%.

Alternatively, the liposoluble active compound may be incorporated into a layer of hydrophobic polymer, for example, in powder or granule form.

In another preferred form, the active substance can include an agent which has a cleansing effect on the skin. Preferably, in this case, the patch comprises a layer which is colored so as to provide the ability to visually quantify and/or qualify the impurities taken up from the skin by the adhesive surface. The presence of the colored layer allows for visual detection of the impurities on the colored surface of the patch, and also allows the impurities to be quantified (in terms of number and size) and/or qualified. The amount of impurities taken up is representative of the desired frequency of application of said patch. Thus, the presence of a large amount of such residues indicates to the user that he or she should apply the patch on the basis of a relatively high frequency (for example every day). A smaller amount of such impurities indicates that the frequency of use can be lower (for example once a week). In addition, the type of impurities taken up enables the user to choose the type of treatment best suited to his or her skin.

The colored pigments can include pigments such as those used in foods or cosmetics, in particular those used for lipsticks or nail varnishes. By way of example, such pigments include, either alone or in combination, synthetic pigments, or mineral pigments, such as zirconium oxide or cerium oxide pigments, as well as iron oxide or chromium oxide and ferric blue. Organic pigments, for example carbon black and barium, strontium, calcium (DC red No. 7) and aluminium lakes also may be used. As a more specific example, a pigment bearing the reference DC violet 2 K7014 sold by Kohnstamm® is used.

The active substance preferably is chosen from keratolytic agents such as _-and _-hydroxycarboxylic or _-keto carboxylic acids, their salts, amides or esters and more particularly _-hydroxy acids such as glycolic acid, lactic acid, tartaric acid, malic acid, citric acid, mandelic acid and fruit acids in general, and _-hydroxy acids such as salicylic acid and its derivatives, preferably its alkyl derivatives, such as 5-n-octanoylsalicylic acid; Kaolin powder, polyamide (Orgasol®) particles, waxes, honey, Sienna, tannins or mineral salts.

The polymer matrix may be a matrix which is self-adhesive (on dry skin and/or on moistened skin) formed, for example, from a polyacrylic or polyvinyl adhesive. A hydrophobic matrix based on a silicone or polyurethane polymer, which is preferably partially crosslinked so as to give it adhesion without the need for an additional adhesive layer, also may be used. An adhesive matrix made of latex, butyl rubber or any other elastomeric adhesive can also be used. In a further aspect, the polymer matrix may be semi-rigid.

As an example, a polyacrylic compound dissolved in a solvent, in particular ethyl acetate or alcohol, is used to make the polymer matrix. The adhesion of the patch can be from about 50 g/cm² to about 800 g/m² (force exerted perpendicular to the plane of the adhesive surface, required to detach it from the skin), and preferably from about 100 g/cm² to about 700 g/cm², even more preferably from about 300 to about 600 g/cm². The surface of the matrix configured to contact with skin may be either smooth or may have bumps or reliefs.

Preferably, the self-adhesive matrix is formed from particles of at least one water-absorbing agent dispersed uniformly throughout the matrix. Specifically, on contact with moisture, especially from the skin, the particles of the water-absorbing agent take up water, thus promoting the dissolution of the water-soluble, solid active compound. By means of this in situ dissolution of the water-soluble active agent, its bioavailability is virtually instantaneous, and any possible interaction with the other compounds present in the polymer layer is therefore minimized. According to some aspects, the polymer matrix, for example, an anhydrous polymer matrix, may be at least partially dissolved, and the magnetic particles may remain substantially immobilized. The skin moisture can act as the agent for dissolving the water-soluble active agent. This effect is even more pronounced if the support layer for the patch and/or the matrix creates occlusive conditions. In a further aspect, the polymer matrix may include one or more charges.

Among the water-absorbing agents which may be present in the hydrophobic polymer matrix in dispersed form, the matrix could include superabsorbent crosslinked polyacrylates with a high degree of swelling in water, such as those sold by the company Norsolor under the name Aquakeep®; polyvinyl alcohol; carboxyvinyl polymers, such as those sold by the Goodrich company under the name Carbopol®; semisynthetic cellulose derivatives, such as carboxymethylcellulose; natural substances, such as starches, natural gums (guar gum, gum arabic, gum tragacanth), casein, phytocolloids (carrageenates, alginates or agar-agar), cotton fibres and gelatin.

It is particularly preferable to use superabsorbent crosslinked polyacrylates, whose presence in dispersed form in a hydrophobic polymer matrix makes it possible to pump up perspiration and promotes, after moistening, better contact with the particles of the other active agents which may be present in the matrix.

The water-absorbing agent, as defined above, preferably is present in a proportion ranging from about 0.2% to about 20% by weight, and more preferably ranging from about 0.5% to about 10% relative to the total weight of the polymer layer.

Alternatively, the matrix may be formed from at least one water-soluble polymer in an amount effective to form a gel upon moistening, the gel adhering to the skin upon drying. Examples of such polymers include polyvinylpyrrolidone (PVP) or polyvinylalcohol (PVA). With such a configuration, the patch can be applied in dry form to moistened skin, or moistened and then applied to dry skin. In general, the application in dry form is preferred.

Alternatively, the polymer matrix can be formed from a gel with a high water content, for example, a hydrogel, preferably based on at least one hydrocolloid and displaying contact adhesion similar to that resulting from a suction-cup effect. Such gels can be either reversible or irreversible gels.

The hydrocolloid(s) used in the patch preferably are chosen, for example, from: gellan gum; cellulose or its derivatives, such as, for example, carboxymethylcellulose, hydroxypropylcellulose, methylcellulose, hydroxypropylmethylcellulose or hydroxyethylcellulose, as well as celluloses modified preferably by grafting an alkyl group; algal extracts such as agar-agar, carrageenans and alginates; seed extracts such as carob gum, guar gum and guar gums modified preferably by grafting an alkyl group; plant exudates such as gum arabic, karaya gum, gum tragacanth and ghatti gum; microorganism exudates such as xanthan gum; fruit extracts such as pectins; gelling agents of animal origin, such as gelatin and caseinates; water-soluble gelling synthetic polymers such as polyacrylic acids optionally crosslinked with an alkyl chain, such as, for example, the Carbopol or Pemulen products from the company Goodrich; silicon derivatives such as synthetic hectorites, for example, the products Laponite RD and Laponite RDS sold by the company Waverly, and aluminium magnesium silicates, for example the product Veegum sold by the company Vanderbilt; and products such as Polycare® sold by the company Rhône-Poulenc, or a mixture of these compounds. Other suitable like materials also may be used.

Preferably, the polymer matrix is deposited on a support. Such a support can be formed of any suitable material which is impermeable to the active compounds contained in the adjacent matrix. The support layer not only supports the matrix but also serves as a protective coating for the matrix. The support layer preferably is the same size as the matrix or larger than it such that it extends beyond the edge of the matrix.

The support layer can be an occlusive support. By way of example, the support layer includes a thermoplastic material chosen from high-density and low-density polyethylenes, polypropylenes, polyvinyl chlorides, copolymers of ethylene and of vinyl acetate, polyesters and polyurethanes. Alternatively, the support layer can include multiple layers (i.e., a complex) of these materials. These materials can also be present in a form laminated with at least one sheet of metal such as an aluminium foil.

The support layer may be of any suitable thickness capable of providing the desired protective and support functions. Preferably, the support layer is from about 20 μm to about 1.5 mm thick. The support layer also should be sufficiently flexible so as to conform to the profile of the skin and not cause the user any sensation of discomfort.

Preferably, however, the support is non-occlusive. In this case, a support formed of a paper, a porous or perforated thermoplastic material, a woven, a nonwoven or a perforated nonwoven is used.

Preferably, the patch according to the invention comprises at least one protective sheet which can be peeled off before the patch is applied. When the polymer matrix is protected with a detachable protective layer, this layer is removed at the time of use. It can be made of any material which is impermeable to the active compound, as well as to any other component present in the polymer matrix. The materials which may be used preferably include, among others, a sheet of silicone paper or a sheet of thermoplastic material treated so as to make it anti-adhesive, for example by using varnish. Preferably, this detachable protective layer is made of polyethylene. Advantageously, the protective sheet is made of two parts superimposed on a middle portion of the patch, so as to facilitate its application without the fingers coming into contact with the matrix containing the active agent(s). According to an alternative, the protective sheet extends over a larger area than the area of the polymer matrix, and passes beyond the limits of this matrix, so as to make it easier to detach.

In a known manner, the patches according to the present invention can be trimmed to a suitable contour corresponding to the region of skin surface to be treated, for example in the shape of a mask for application to the face, in particular for application to the nose, the lips, the cheeks, the area between the nose and the top lip, or to the forehead. Other shapes required for application to a given region of the body also are contemplated, for example, to the thighs for a patch with a weight-reducing action or to the bust for a patch with a firming action. In general, the size of a patch in accordance with the invention is between about 0.25 cm² and about 500 cm², and preferably between about 1 cm² and about 30 cm².

The patch preferably is packaged in a tray or in a protective sachet formed from a two sheet layered structure (i.e., complex), such as including a paper layer and a film layer made of leakproof plastic. The paper can be coated with a cold-sealable adhesive and the sheets can be sealed around the patch by contact with the faces coated with such adhesive.

Preferably, such a patch is used for a relatively short application time, as compared to conventional patches. Its application time is preferably from about 30 seconds to about 15 minutes, and even more preferably from about 30 seconds to about 5 minutes.

In a preferred form, the patch according to the invention comprises at least one region of small size, in the shape of a wedge, capable of helping to detach the patch.

A frame, either woven or nonwoven polyamide for example, can be at least partially immersed in the matrix. The frame can act as a support, and vice versa. It gives the patch greater cohesion, in particular when the matrix is of the type forming a gel. In addition, in the case of a self-adhesive matrix, the frame makes it possible to vary the adhesion of the patch to the skin. Preferably, some of the magnetic particles will be contained directly in the frame, which is thus magnetizable, which may render the frame able to produce a magnetic field. Such a characteristic may be particularly advantageous if one of the active substances is incompatible with certain magnetic particles should they be directly incorporated into the matrix.

According to another aspect of the invention, a process for manufacturing a patch includes forming a polymer matrix. One surface of the matrix is intended to be placed in contact with the skin, and this surface is adhesive or capable of becoming adhesive, preferably in the presence of water. The matrix includes at least one active substance capable of treating or reacting with the skin, in particular cosmetically treating or reacting with the skin. At least one magnetic particle is in the matrix. Preferably, a plurality of magnetic particles are dispersed uniformly throughout the matrix. The process further includes placing the polymer matrix in a magnetic field so as to permanently orient the magnetic particle(s), for example, over at least a portion of the area of the patch. Preferably, the placing in the magnetic field occurs after at least partial polymerization of the matrix. According to another aspect, a patch may be formed without undergoing any aqueous phase. In yet another aspect, the magnetic particles may be disposed randomly within the polymer matrix and their distribution within the polymer matrix may be substantially homogeneous, for example, such that the magnetic field may be substantially constant before and after a first use.

The polymer matrix can be magnetized in various ways. As a non-limiting example, magnetization devices of the type including windings with or without soft iron are used. Pulsed current created by a magnetization bed flows through the windings of the magnetization device to generate the magnetic field required to magnetize the matrix. Magnetization beds, such as those featured in the catalogue from the company TE2M® (Techniques et Materiels Magnetiques) under the references CE 500, PM 1000 or PM 2500, which are medium- to high-power devices, can be used, making it possible to carry out a high rate of magnetization of magnets of various grades and shapes.

Preferably, the process includes depositing the matrix continuously onto a strip of support, conveying the support coated with the polymer matrix through a magnetic field (for example, after at least partial polymerization of the matrix), cutting the magnetized matrix and support into the form of patches of the desired shape and size, and placing the cut up patches in protective sachets.

In another aspect of the invention, the process includes depositing the matrix continuously on a strip of support, cutting up the support coated with the matrix into the form of patches of desired shape and size, conveying the patches thus formed through a magnetic field (for example, after at least partial polymerization of the matrix), and placing the magnetized patches in protective sachets.

Yet another aspect according to the invention includes a process comprising depositing the matrix continuously onto a strip of support, cutting up the support coated with the matrix into the form of patches of the desired shape and size, placing the cut up patches in protective sachets, and conveying the sachets through a magnetic field.

When the polymer matrix is formed from a gel, such as, for example, an aqueous gel formed from at least one hydrocolloid, the process may include pouring the matrix in liquid form into a tray of the shape and size of the patch to be made, and in which a frame may optionally be placed, with the matrix being poured into the tray via an aperture formed by one edge of said tray. The process further includes bringing about the at least partial polymerization of the matrix, for example by crosslinking, polyaddition, or polycondensation, and subjecting the tray to a magnetic field capable of permanently orienting the magnetic particles of the polymer matrix.

The aperture of the tray may then be closed by means of a removable protective foil. Alternatively, the tray can be introduced in open form into a protective sachet.

Yet another aspect of the invention includes a patch for treating the skin which comprises at least one polymer matrix having a surface which is adhesive or capable of becoming adhesive and configured to be placed in contact with the skin, wherein said matrix includes at least one active substance capable of acting on the skin and a plurality of magnetic particles homogeneously dispersed in the matrix and permanently oriented over at least a portion of the patch.

Another aspect of the invention includes a patch assembly for treating the skin, comprising a polymer matrix including at least one active substance for acting on the skin and a support covering one surface of the polymer matrix. At least one magnetic particle is in the matrix and this particle is permanently oriented. A surface of the polymer matrix opposite to the support surface is configured to be adhered to the skin.

Another aspect of the invention includes a process for treating the skin comprising providing a polymer matrix having a surface configured to adhere to the skin. The polymer matrix includes an active substance adapted to act on the skin and at least one permanently oriented magnetic particle dispersed in the polymer matrix. The process further includes placing the polymer matrix in contact with a portion of the skin to be treated and adhering the polymer matrix to the skin for a time period sufficient to treat the skin.

Aside from the structural and procedural arrangements set forth above, the invention can include a number of other arrangements, such as those explained hereinafter. It is to be understood that both the foregoing description and the following description are exemplary, and are intended to provide further explanation of the invention as claimed.

The accompanying drawings are included to provide a further understanding of the invention and are incorporated in and constitute a part of this specification. The drawings illustrate embodiments of the invention and, together with the description, serve to explain the principles of the invention. In the drawings,

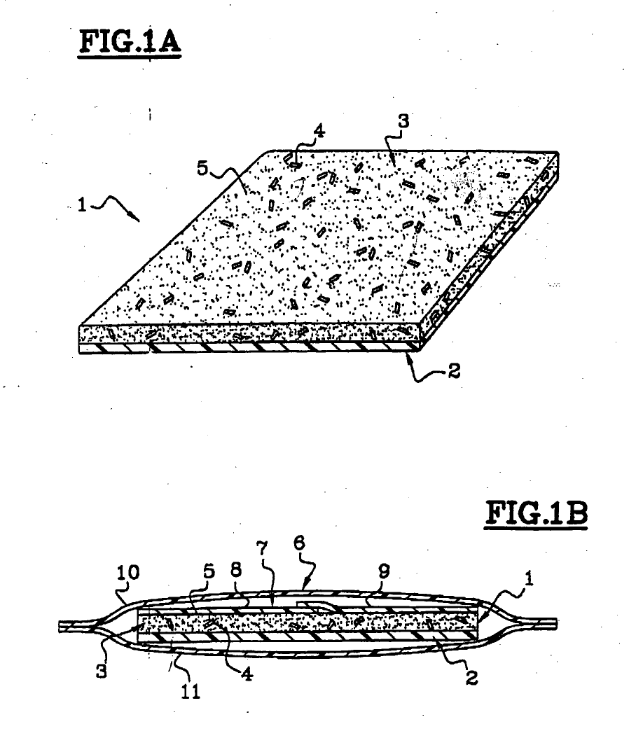

FIG. 1A is a perspective view of one embodiment of a patch according to the present invention;

FIG. 1B is a cross-sectional view of the patch shown in FIG. 1A, showing the patch in its packaged form;

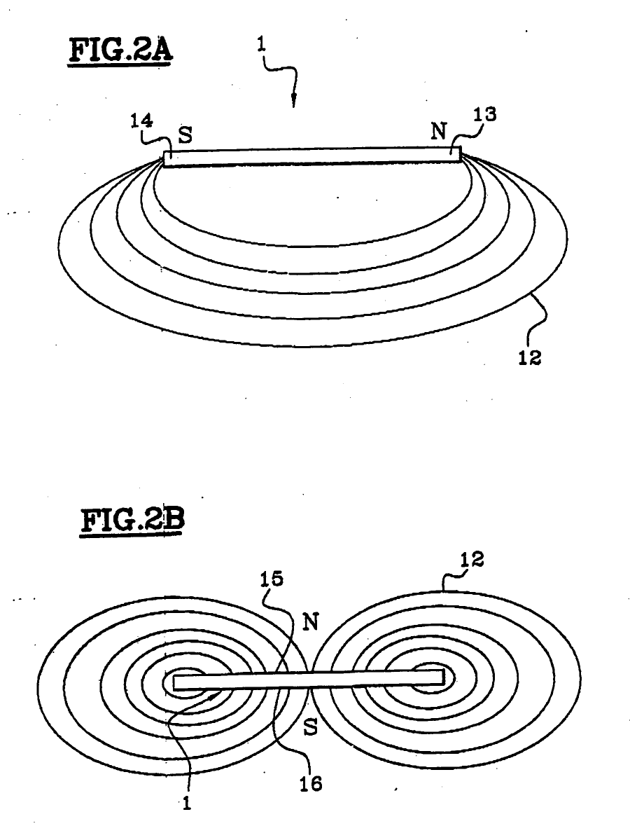

FIGS. 2A and 2B are two examples of magnetization patterns which can be obtained with a patch according to the present invention;

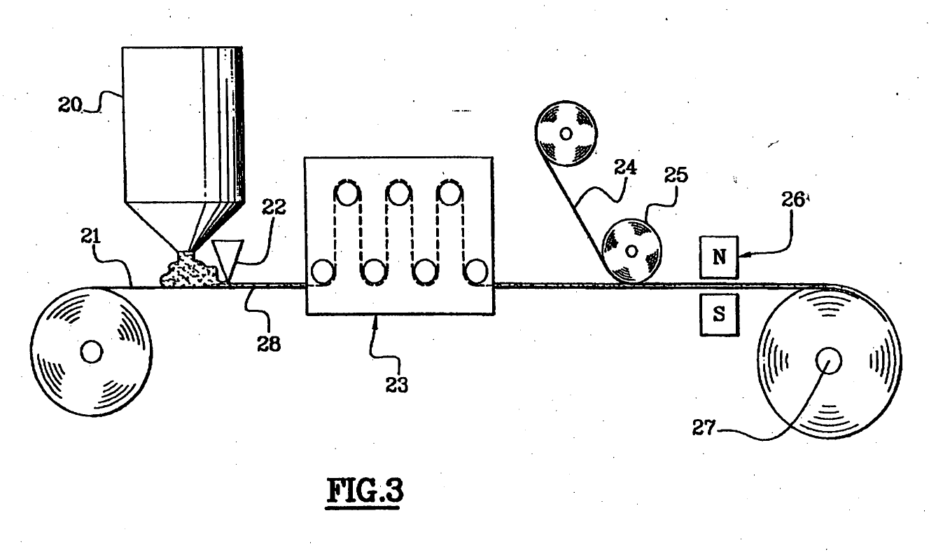

FIG. 3 is a schematic view of one embodiment of a process for manufacturing a patch according to the present invention;

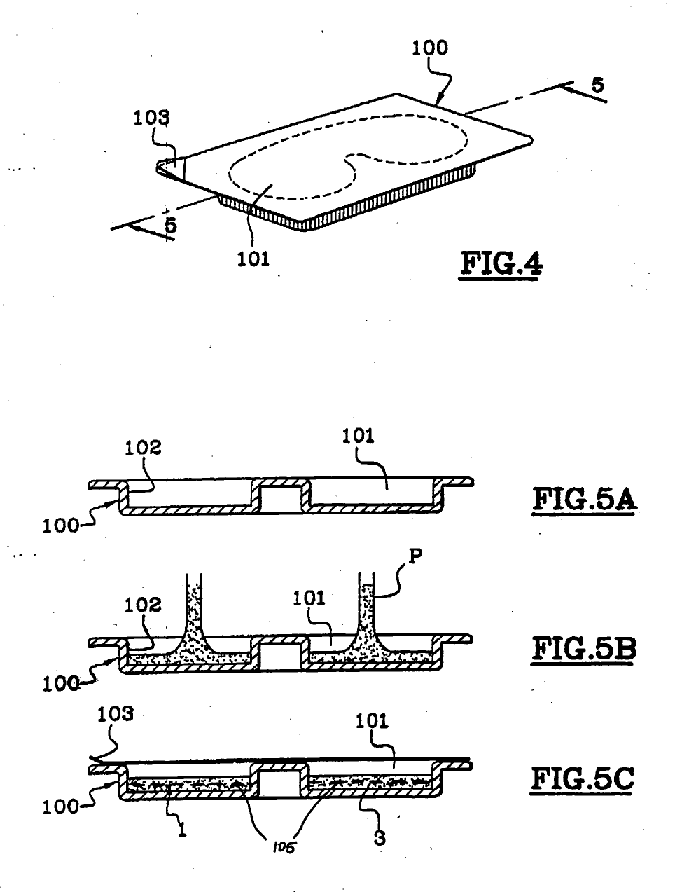

FIG. 4 is a perspective view of another embodiment of a patch according to the invention; and

FIGS. 5A-5C are cross-sectional views of the patch of FIG. 4 showing a process for manufacturing and packaging such a patch.

Reference will now be made in detail to the present preferred embodiments of the invention, examples of which are illustrated in the accompanying drawings. Wherever possible, the same reference numbers are used in the drawings and the description to refer to the same or like parts.

The patch 1 shown in FIG. 1A comprises a polyethylene support 2 on which is deposited a matrix 3 comprising at least one active substance which treats or reacts with the skin, in particular achieving a cosmetic effect. The matrix 3 includes magnetic particles 4 which, as will be explained in greater detail hereinbelow, are magnetically permanently oriented. The face 5 of the matrix 3, which is opposite the support 2, is configured to come into contact with the skin and adhere thereto.

The adhesion of the face 5 of the matrix 3 to the skin can result from the use of a self-adhesive matrix formed, for example, from an acrylic or vinyl polymer. Such a matrix can be self-adhesive either when dry or upon placement on moistened skin or upon being moistened in some other manner. In the case of a matrix that becomes adhesive after being placed on moistened skin, water-absorbing agents capable of drying the skin locally and of forming points for attaching the adhesive matrix to the skin preferably will be incorporated into the matrix. In one alternative, the adhesion of face 5 of the matrix 3 to the skin can result from the preparation of the matrix as a hydratable gel, formed, for example, from PVP or PVA. Such a matrix adheres to the skin on becoming dry. In another alternative, the adhesion of face 5 of the matrix 3 to the skin can result from a suction-cup effect resulting from the preparation of the matrix in the form of a gel with a high water content, comprising at least one hydrocolloid, such as, for example, gellan gum.

In FIG. 1B, the patch 1 is placed inside a protective sachet 6. Prior to such placement, the face 5 of the matrix may be precoated with a peelable foil 7, preferably comprising two portions 8 and 9 which overlap in a mid-zone of the patch 1. The protective sachet 6 is formed from two sheets 10, 11 of a paper/film complex, the film layer being made of leakproof plastic and the paper preferably being coated with a cold-sealable adhesive. The sheets are sealed around the patch 1 by contact with the faces coated with adhesive.

Referring to FIGS. 2A and 2B, by way of example, two patterns of magnetization of the patch 1 according to the present invention are shown. In FIG. 2A, the patch is magnetized such that the magnetic north (N) is located at one end 13 of the patch 1, while the magnetic south (S) is located at a second end 14 of the patch 1, opposite the first end. The lines of magnetic flux 12 extend in the manner shown in FIG. 2A.

In FIG. 2B, the patch is magnetized such that the magnetic north (N) is located on a first face 15 of the patch 1, while the magnetic south (S) is located on a second face 16 of the patch 1, opposite the first face. The lines of magnetic flux 12 extend in the manner shown in FIG. 2B. A multitude of different patterns can be obtained depending on such factors as, for example, the configuration of the magnetic field(s) in which the patch is placed, and/or of the number of times the patch 1 is placed in such a magnetic field. According to another alternative, the ferromagnetic particles can be oriented such that a first polarity is central, while the other is arranged in a ring around the first polarity.

FIG. 3 shows one possible process for manufacturing a patch according to the present invention. According to a preferred method of the invention, a mixture is prepared using a polymer, preferably an acrylic polymer, dissolved in a solvent such as an alcohol, for example, ethanol or ethyl acetate. The active agent(s) and the ferromagnetic particles are then added to the mixture.

The mixture is introduced into a hopper 20 and poured onto a sheet of silicone paper, or treated polyethylene 21, forming the detachable or peelable protective foil of the patch. Downstream of the hopper 20 is located a scraper 22 for equalizing the thickness of the polymer layer 28 of the polymer matrix. This thickness preferably is between about 10 μm and about 300 μm.

The solvents preferably are made to evaporate at a temperature of from about 30° C. to about 100° C., by passing the layer 28 thus formed on the sheet 21 into an oven 23. An adhesive layer is thus obtained and a support 24, preferably a nonwoven support is deposited on the adhesive layer 28. The assembly is then calendered by means of a calender roller 25, and is conveyed continuously across a magnetization bed 26, so as to permanently orient the ferromagnetic particles contained in the polymer layer 28. The assembly thus formed is then wound on a core 27.

The entire assembly is then passed through a cutting station to cut it into the desired shape and size, thus forming individual patches. Prior to the cutting step, the sheet 21 of silicone paper or treated polyethylene, which served to convey the polymer layer 28 through the various stations of the device mentioned above can be replaced with a structure containing two sheets which overlap in a mid-zone. After being cut, the patches may then be placed in individual sachets. In an alternative manner, the patches may also be magnetized after placing them in sachets.

FIG. 4, to which reference is now made, shows a tray 100 defining a compartment 101 inside which a patch is molded according to yet another embodiment of the invention. According to this embodiment, the matrix preferably is formed from a hydrocolloid, such as, for example, a gellan gum, or like substance. In the same way as was discussed with reference to FIGS. 1A and 1B, the matrix comprises ferromagnetic particles. The tray 100 is obtained by a technique, such as thermoforming or thin-wall injection, of a material such as a polypropylene. The compartment 101 has a shape corresponding to that of a mask for the eyes. The compartment 101 includes an edge delimiting an aperture, which can be closed off by a heat-sealed cover 103.

FIGS. 5A-5C illustrate a view in cross-section along the line 5-5 of the assembly in FIG. 4. In FIG. 5A, the tray 100 is placed on its base, with the aperture 102 of the compartment 101 being open. In FIG. 5B, the liquid composition P capable of forming the gelled matrix 3 of the patch 1 is poured into the compartment 101 via the aperture 102. In this pouring phase, the temperature of the liquid composition P preferably is about 90° C.

As shown in FIG. 5C, a frame 105 could be added to the interior of the tray 100 before complete setting of the matrix. The frame preferably becomes at least partially embedded in the matrix to form a part of the patch.

During cooling of the composition P, the liquid composition sets so as to form a gelled patch 1. Preferably, the setting takes place at a temperature which can be from about 60° C. to 70° C. Before, during, or after the setting of the composition, a cover 103 can be heat-sealed so as to close off the aperture 102. Before or after closing the tray, it can be conveyed across a magnetization bed of the type used in the preceding embodiment, so as to permanently orient the magnetic particles contained in the matrix 3.

### EXAMPLE I

A composition is prepared comprising, on a volume basis:
80.5% acrylic polymer dissolved in ethyl acetate;
1.5% salicylic acid;
2% caffeine;
3% vitamin C;
8% polyacrylate; and
5% ferrite particles.

After homogenization, the product is introduced into a hopper and is spread out using a scraper into a layer 0.8 mm thick over a sheet of polyethylene 200 μm thick. This sheet can be pretreated at the surface to reduce its adhesion. The assembly is heated to 60° C. so as to evaporate the solvent. A nonwoven formed from 30% polyethylene terephthalate and 70% viscose is then applied to the free surface of the polymer layer. This nonwoven constitutes the support layer for the patch. The entire assembly is then calendared. An assembly is thus obtained comprising a support layer and a layer of self-adhesive polymer formed from a polyacrylic matrix, this assembly also comprising a detachable protective layer. The assembly thus formed is conveyed into a magnetization bed, such as a PM 1000 marketed by the company TE2M®, and then cut into the desired shape and size. After cutting, the patches are packaged in polyethylene sachets.

Such a patch made according to Example 1 is particularly effective for treating wrinkles and bags around the eyes, the penetration of the active agents in the patch being promoted by the presence of permanently magnetized ferrite particles, which have an action on the circulation in the capillaries in the region to which the patch is applied.

### EXAMPLE II

A composition is prepared comprising, on a volume basis:
77.5% acrylic polymer dissolved in ethyl acetate;
3% glycerol;
10% ferrite;
5% cotton fibers;
0.5% kojic acid;
3% caffeine; and
1% green tea.

The procedure for forming the patch corresponds essentially to that described in the above Example I.

Such a patch has a relaxing, lightening and tiredness-relieving effect, the action of the active agents being reinforced by the presence of permanently magnetized ferrite particles, which contribute to making the skin more receptive to such active agents.

Preferably, the patch of the present invention contains a cosmetic, pharmaceutical, or dermo-pharmaceutical product. In its broadest aspects, the present invention could be used to apply other types of substances to achieve other effects within the body, for example on muscles and the like.

Sizes of various structural parts and materials used to make the above-mentioned parts are illustrative and exemplary only, and one of ordinary skill in the art would recognize that these sizes and materials can be changed to produce different effects or desired characteristics.

It will be apparent to those skilled in the art that various modifications and variations can be made to the structure and methodology of the present invention. Thus, it should be understood that the invention is not limited to the examples discussed in the specification. Rather, the present invention is intended to cover modifications and variations.

1. A patch comprising:
at least one polymer matrix having a surface intended to be placed in contact with the skin, the surface being one of adhesive and configured to become adhesive,
wherein the polymer matrix comprises at least one active substance effective for acting on the skin, the at least one active substance comprising a solid active substance, and
wherein the polymer matrix is anhydrous; and
a plurality of magnetic particles in the polymer matrix, the plurality of magnetic particles being permanently oriented over at least a portion of the patch,
wherein the patch is in dry form prior to use.

2. The patch of claim 1, wherein the plurality of magnetic particles include ferrite.

3. The patch of claim 1, wherein the percentage by volume of the plurality of magnetic particles in the polymer matrix ranges from about 0.2% to about 80%.

4. The patch of claim 1, wherein the percentage by volume of the plurality of magnetic particles in the polymer matrix ranges from about 1% to about 30%.

5. The patch of claim 1, wherein the percentage by volume of the plurality of magnetic particles in the polymer matrix ranges from about 2% to about 15%.

6. The patch of claim 1, wherein the plurality of magnetic particles are coated with one of polyurethane, epoxy resin, polyester, polyamide, and cyanoacrylate.

7. The patch of claim 1, wherein the plurality of magnetic particles are oriented such that generated magnetic field lines extend, over at least a portion of the patch, from a first face of the patch to a second face of the patch opposite the first face.

8. The patch of claim 1, wherein the plurality of magnetic particles are oriented such that generated magnetic field lines extend, over at least a portion of the patch, from a first edge of the patch to a second edge of the patch opposite the first edge.

9. The patch of claim 1, wherein the at least one active substance is chosen from weight-reducing agents, cleansing agents, antioxidants, free-radical scavengers, moisturizers, depigmenting agents, liporegulators, anti-acne agents, anti-seborrhoeic agents, anti-ageing agents, softeners, anti-wrinkle agents, keratolytic agents, anti-inflammatory agents, refreshing agents, cicatrizing agents, vascular protectors, antibacterial agents, antifungal agents, antiperspirants, deodorants, skin conditioners, desensitizers, immunomodulators and nourishing agents, moisture absorbers, and sebum absorbers.

10. The patch of claim 9, wherein the at least one active substance is a cleansing agent and said patch further comprises a layer which is colored so as to provide visibility of impurities taken up from the skin by the adhesive surface.

11. The patch of claim 1, wherein the at least one active substance is a keratolytic agent chosen from α- and β-hydroxycarboxylic and β-keto carboxylic acids, their salts, amides, esters, Kaolin powder, polyamide particles, waxes, honey, Sienna, tannins, and mineral salts.

12. The patch of claim 11, wherein the α-hydroxycarboxylic acid is chosen from glycolic acid, lactic acid, tartaric acid, malic acid, citric acid, mandelic acid, and fruit acids.

13. The patch of claim 11, wherein the β-hydroxycarboxylic acid is chosen from salicylic acid and its derivatives.

14. The patch of claim 1, wherein the at least one active substance is a water-soluble active substance chosen from enzymes; antibiotics; components with a tensioning effect; sugars and sugar derivatives; urea; oligopeptides; water-soluble plant extracts; yeasts; protein hydrolysates; mucopolysaccharides; vitamins B₂, B₆, H, and PP; panthenol; allantoin; hydroquinone; α-hydroxy acids; polyhydroxy acids; amino acids; hyaluronic acid; folic acid; acetylsalicylic acid; glycyrrhetic acid; kojic acid; ascorbic acid; and biologically compatible salts thereof.

15. The patch of claim 1, wherein the at least one active substance is a liposoluble active substance chosen from D-α-tocopherol, DL-α-tocopherol, D-α-tocopheryl acetate, DL-α-tocopheryl acetate, ascorbyl palmitate, vitamin F and vitamin F glycerides, D vitamins, vitamin D₂, vitamin D₃, retinol, retinol esters, retinyl palmitate, retinyl propionate, β-carotene, D-panthenol, farnesol, farnesyl acetate, jojoba oil and blackcurrant oil rich in essential fatty acids, keratolytic agents, including salicylic acid, its salts and its esters, 5-n-octanoylsalicylic acid and its esters, alkyl esters of α-hydroxy acids including one of citric acid, lactic acid and glycolic acid, asiatic acid, madecassic acid, asiaticoside, total extract of Centella asiatica, β-glycyrrhetinic acid, α-bisabolol, ceramides, including 2-oleoylamino-1,3-octadecane, phytanetriol, milk sphingomyelin, phospholipids of marine origin rich in polyunsaturated essential fatty acids, ethoxyquine, extract of rosemary, extract of balm, quercetin, extract of dried microalgae, and steroidal anti-inflammatory agents.

16. The patch of claim 1, wherein the polymer matrix is self-adhesive and comprises at least one of an acrylic polymer, a vinyl polymer, a silicone, a polyurethane, a latex, and a butyl rubber.

17. The patch of claim 16, wherein the polymer matrix further comprises at least one water-absorbing agent chosen from superabsorbent crosslinked polyacrylates, polyvinyl alcohol, carboxyvinyl polymers, semisynthetic cellulose derivatives, starches, guar gums, gum arabic, gum tragacanth, casein, phytocolloids, cotton fibres, and gelatin.

18. The patch of claim 1, wherein the polymer matrix comprises at least one water-soluble polymer in an amount effective to form a gel after moistening that adheres to the skin upon drying.

19. The patch of claim 18, wherein the water-soluble polymer is chosen from polyvinylpyrrolidone and polyvinylalcohol.

20. The patch of claim 1, wherein the polymer matrix is deposited on a support.

21. The patch of claim 20, wherein the support is an occlusive support comprising at least one thermoplastic material chosen from high-density polyethylenes, low-density polyethylenes, polypropylenes, polyvinyl chlorides, copolymers of ethylene, copolymers of vinyl acetate, polyesters, polyurethanes, and multiple layers thereof.

22. The patch of claim 20, wherein the support is a non-occlusive support comprising at least one material chosen from a paper, a porous thermoplastic material, a perforated thermoplastic material, a woven material, a nonwoven material, and a perforated nonwoven material.

23. The patch of claim 1, further comprising at least one protective sheet configured to be peeled off before the patch is placed in contact with the skin.

24. The patch of claim 23, wherein the protective sheet includes two parts that superimpose each other over a mid-portion of the patch.

25. The patch of claim 1, further comprising a frame comprising at least one material chosen from a woven material and a nonwoven material, said frame being at least partially embedded in the polymer matrix.

26. The patch of claim 25, wherein the plurality of magnetic particles is in the frame.

27. A packaged patch system, comprising:
the patch of claim 1; and
a protective sachet formed from of a layer of paper and a layer of film comprising leakproof plastic, the sachet being sealed around the patch.

28. The patch of claim 1, wherein the polymer matrix comprises at least one water-soluble agent.

29. The patch of claim 1, wherein polymer matrix is semi-rigid.

30. The patch of claim 1, wherein the polymer matrix comprises charges.

31. The patch of claim 1, wherein the polymer matrix comprises at least one water-absorbing agent.

32. The patch of claim 1, wherein the polymer matrix is at least partially soluble in liquid.

33. The patch of claim 32, wherein the plurality of magnetic particles remain permanently oriented when the polymer matrix is partially dissolved in liquid.

34. The patch of claim 1, wherein the polymer matrix comprises a gel, the plurality of magnetic particles being mixed in the gel such that the plurality of magnetic particles are distributed in the polymer matrix.

35. The patch of claim 1, wherein the plurality of magnetic particles are randomly disposed within the polymer matrix in a substantially homogeneous manner such that the plurality of magnetic particles generate a magnetic field that remains substantially constant before and after a first use.

36. The patch of claim 1, wherein the patch is configured to generate a magnetic field having a strength ranging from about 0.3 gauss to about 10 gauss.

37. The patch of claim 1, wherein the patch is configured such that when saturated with water, the patch increases circulation.

38. The patch of claim 1, further comprising a frame associated with the polymer matrix, wherein the frame is configured to produce a magnetic field.

39. A patch assembly for treating the skin, the patch assembly comprising:
a polymer matrix comprising at least one active substance for acting on the skin, the at least one active substance comprising a solid active substance,
wherein the polymer matrix is anhydrous;
a support surface at least partially covering one surface of the polymer matrix; and
a plurality of magnetic particles in the polymer matrix, the plurality of magnetic particles being permanently oriented over at least a portion of the patch assembly,
wherein a surface of the polymer matrix opposite to the support surface is configured to be adhered to the skin, and
wherein the patch assembly is in dry form prior to use.

40. The assembly of claim 39, wherein the plurality of magnetic particles are uniformly dispersed throughout the matrix.

41. The assembly of claim 39, further comprising a removable protective sheet disposed on a surface of the polymer matrix opposite the support surface, wherein the protective sheet is configured to be removed prior to applying the assembly to the skin.

42. The assembly of claim 39, further comprising a protective sachet formed from a layer of paper and a layer of film comprising leakproof plastic, the sachet being sealed around the support and polymer matrix.

43. The assembly of claim 39, wherein the support is an occlusive support comprising at least one thermoplastic material chosen from high-density polyethylenes, low-density polyethylenes, polypropylenes, polyvinyl chlorides, copolymers of ethylene, copolymers of vinyl acetate, polyesters, polyurethanes, and multiple layers thereof.

44. The assembly of claim 39, wherein the support is a non-occlusive support comprising at least one material chosen from a paper, a porous thermoplastic material, a perforated thermoplastic material, a woven material, a nonwoven material, and a perforated nonwoven material.

45. The assembly of claim 39, wherein the polymer matrix comprises at least one water-soluble polymer in an amount effective to form a gel after moistening that adheres to the skin upon drying.

46. The assembly of claim 39, wherein the polymer matrix is self-adhesive and comprises at least one of an acrylic polymer, a vinyl polymer, a silicone, a polyurethane, a latex, and a butyl rubber.

47. The assembly of claim 39, wherein the at least one active substance is chosen from weight-reducing agents, cleansing agents, antioxidants, free-radical scavengers, moisturizers, depigmenting agents, liporegulators, anti-acne agents, anti-seborrhoeic agents, anti-ageing agents, softeners, anti-wrinkle agents, keratolytic agents, anti-inflammatory agents, refreshing agents, cicatrizing agents, vascular protectors, antibacterial agents, antifungal agents, antiperspirants, deodorants, skin conditioners, desensitizers, immunomodulators and nourishing agents, moisture absorbers, and sebum absorbers.

48. The assembly of claim 47, wherein the at least one active substance is a cleansing agent, and said assembly further comprises a layer that is colored so as to provide visibility of impurities taken up from the skin by the adhesive matrix.

49. The assembly of claim 39, wherein the at least one active substance is a keratolytic agent chosen from α- and β-hydroxycarboxylic and β-keto carboxylic acids, their salts, amides or esters, Kaolin powder, polyamide particles, waxes, honey, Sienna, tannins, and mineral salts.

50. The assembly of claim 49, wherein the α-hydroxycarboxylic acid is chosen from glycolic acid, lactic acid, tartaric acid, malic acid, citric acid, mandelic acid, and fruit acids.

51. The assembly of claim 49, wherein the β-hydroxycarboxylic acid is chosen from salicylic acid and its derivatives.

52. The assembly of claim 39, wherein the at least one active substance is a water-soluble active substance chosen from enzymes; antibiotics; components with a tensioning effect; sugars and sugar derivatives; urea; oligopeptides; water-soluble plant extracts; yeasts; protein hydrolysates; mucopolysaccharides; vitamins B₂, B₆, H, and PP; panthenol; allantoin; hydroquinone; α-hydroxy acids; polyhydroxy acids; amino acids; hyaluronic acid; folic acid; acetylsalicylic acid; glycyrrhetic acid; kojic acid; ascorbic acid; and biologically compatible salts thereof.

53. The assembly of claim 39, wherein the at least one active substance is a liposoluble active substance chosen from D-α-tocopherol, DL-α-tocopherol, D-α-tocopheryl acetate, DL-α-tocopheryl acetate, ascorbyl palmitate, vitamin F and vitamin F glycerides, D vitamins, vitamin D₂, vitamin D₃, retinol, retinol esters, retinyl palmitate, retinyl propionate, β-carotene, D-panthenol, farnesol, farnesyl acetate, jojoba oil and blackcurrant oil rich in essential fatty acids, keratolytic agents, salicylic acid, its salts and its esters, 5-n-octanoylsalicylic acid and its esters, alkyl esters of α-hydroxy acids, citric acid, lactic acid, glycolic acid, asiatic acid, madecassic acid, asiaticoside, total extract of Centella asiatica, β-glycyrrhetinic acid, α-bisabolol, ceramides, 2-oleoylamino-1,3-octadecane, phytanetriol, milk sphingomyelin, phospholipids of marine origin rich in polyunsaturated essential fatty acids, ethoxyquine, extract of rosemary, extract of balm, quercetin, extract of dried microalgae, and steroidal anti-inflammatory agents.

54. The assembly of claim 39, wherein the polymer matrix further comprises at least one water-absorbing agent chosen from superabsorbent crosslinked polyacrylates, polyvinyl alcohol, carboxyvinyl polymers, semisynthetic cellulose derivatives, starches, guar gums, gum arabic, gum tragacanth, casein, phytocolloids, cotton fibers, and gelatin.

55. A process for treating the skin, comprising:
providing a patch comprising
a polymer matrix comprising
a surface configured to adhere to the skin, and
an active substance adapted for acting on the skin, the active substance comprising a solid active substance,
wherein the polymer matrix is anhydrous, and
a plurality of permanently oriented magnetic particles dispersed over at least a portion of the polymer matrix,
wherein the patch is in dry form prior to use;
placing the patch in contact with a portion of the skin to be treated; and
adhering the patch to the skin for a time period sufficient to treat the skin.

56. The process of claim 55, wherein the active substance includes a cleansing agent and treating the skin includes cleansing the skin.

57. The process of claim 55, wherein a magnetic field generated by the plurality of magnetic particles acts on circulation in capillaries in the portion of the skin to which the patch is applied.

58. The process of claim 55, wherein the polymer matrix is adhered to the skin by moistening the polymer matrix.

59. The process of claim 55, wherein the acting on the skin is accomplished by an active substance chosen from weight-reducing agents, cleansing agents, antioxidants, free-radical scavengers, moisturizers, depigmenting agents, liporegulators, anti-acne agents, anti-seborrhoeic agents, anti-ageing agents, softeners, anti-wrinkle agents, keratolytic agents, anti-inflammatory agents, refreshing agents, cicatrizing agents, vascular protectors, antibacterial agents, antifungal agents, antiperspirants, deodorants, skin conditioners, desensitizers, immunomodulators and nourishing agents, moisture absorbers, and sebum absorbers.

60. A patch for treating the skin, comprising:
at least one polymer matrix having a surface configured to be placed in contact with and adhered to the skin, wherein the at least one polymer matrix comprises
at least one active substance effective for acting on the skin, the at least one substance active substance comprising a solid active substance,
wherein the polymer matrix is anhydrous; and
a plurality of magnetic particles homogeneously dispersed in the matrix, said magnetic particles being permanently oriented over at least a portion of the patch,
wherein the patch is in dry form prior to use.
